(19) United States
(12) Patent Application Publication
(10) Pub. No.: US 2023/0414567 A1
(43) Pub. Date: Dec. 28, 2023

(54) METHODS OF TREATING CONDITIONS RELATED TO THE S1P1 RECEPTOR

(71) Applicant: Arena Pharmaceuticals, Inc., New York, NY (US)

(72) Inventors: Gurpreet AHLUWALIA, Santa Ana, CA (US); Maple M FUNG, San Diego, CA (US); Andrew Christopher Wesley SELFRIDGE, Buckinghamshire (GB)

(21) Appl. No.: 18/250,596

(22) PCT Filed: Nov. 8, 2021
(86) PCT No.: PCT/US2021/058483

§ 371 Date: Apr. 26, 2023

Related U.S. Application Data
(60) Provisional application No. 63/111,312, filed on Nov. 9, 2020.

(30) Foreign Application Priority Data
Nov. 9, 2020 (US) 63111312

(51) Int. Cl. A61K 31/403; A61P 17/04

(52) U.S. Cl. CPC A61K 31/403; A61P 17/04

(57) ABSTRACT

Described herein are methods for treating atopic dermatitis that allows for the effective treatment of the disease with an interruption period if certain adverse events are observed. Additionally, described here are new methods of treating moderate to severe atopic dermatitis.

Provided are methods useful in the treatment of sphingosine 1-phosphate subtype 1 (SIP₁ or SIP1) receptor-associated disorders, and more particularly atopic dermatitis. Atopic dermatitis (AD, also known as atopic eczema) is the most common chronic relapsing, inflammatory skin disease. AD is a serious, chronic immune-mediated disease in which symptoms vary, but often include severe dry skin, itching, patches, swollen skin and raised bumps which may leak fluid. The lifetime prevalence of AD is 10% to 20% in developed nations, and appears to be increasing (Weidinger 2016, Heratizadeh 2017). AD can remain a chronic and lifelong condition, with prevalence in adults ranging from 7% to 10% (Boguniewicz 2017). In a clinical study population, it was found that 50% of AD patients had been living with active disease for more than 27 years (Simpson 2016b).

AD is characterized by systemic cutaneous inflammation and perturbed epidermal-barrier function resulting in dry, red skin and intense itch (Bieber 2008, Weidinger 2016). Essential features for diagnosis of AD are pruritus, eczematous dermatitis, and a chronic or relapsing history of disease (Weidinger 2016, Boguniewicz 2017). Patients with AD are more likely to have other allergic or atopic conditions. In a 2016 study of 380 adults with moderate-to-severe AD, 51.3% had allergic rhinitis, 40.3% had asthma, 24.2% had allergic conjunctivitis, and 60.5% had other allergies. The disease burden of those with AD significantly impacts quality of life and patients report that their condition affects social and leisure activities (43.9%), work or studying (41.8%), and even clothing choice (57.9%) (Simpson 2016).

A combination of genetic, environmental, and immunologic factors appears to determine disease predisposition, while the pathogenesis of AD is thought to stem from the mutually reinforcing interaction between a disrupted epidermal barrier and an inappropriate immune response in the skin (Weidinger 2016, Heratizadeh 2017). Epidermal barrier disruption in AD facilitates penetration by allergens, immunoglobulin E (IgE) sensitization, and bacterial colonization (particularly Staphylococcus aureus), which induce persistent type 2 helper T cell responses (Salava 2014, Zhu 2018).

There are currently various topical and systemic therapeutic options that are used for the treatment and symptomatic relief of AD, including corticosteroids, moisturizers, and systemic immunosuppressants. However, these current therapies are limited by poor compliance, safety profiles that prohibit long-term use, limited efficacy that provides only transient or marginal symptomatic relief, invasive administration procedures, or off-label use. Thus, there remains a great unmet medical need for an effective, safe, and orally administered treatment.

There also remains a need for an effective treatment and treatment methods that balance risks associated with S1P receptor modulators, such as the known effect of lymphocyte lowering in patients receiving S1P receptor modulators.

### SUMMARY

Applicant has discovered new methods of treating moderate to severe atopic dermatitis, including methods that allow for the effective treatment of the disease with an interruption period if certain biomarkers or adverse events are observed.

Three SIP modulators are approved for the treatment of relapsing forms of multiple sclerosis—fingolimod (GILENYA), siponimod (MAYZENT), and ozanimod (ZEPOSIA) (MAYZENT (siponimod) (2020) Prescribing Information. Novartis AG., in; MAYZENT (siponimod) (2020) Prescribing Information. Novartis AG., in; ZEPOSIA (ozanimod) (2020) Prescribing Information; Celgene, in). Ozanimod is also in clinical development for ulcerative colitis and Crohn's disease (Peyrin-Biroulet L, Christopher R, Behan D and Lassen C (2017) Modulation of sphingosine-1-phosphate in inflammatory bowel disease. Autoimmun Rev 16:495-503). Etrasimod has demonstrated efficacy in a Phase 2 clinical trial in ulcerative colitis (Sandborn W J, Peyrin-Biroulet L, Zhang J, Chiorean M, Vermeire S, Lee S D, Kühbacher T, Yacyshyn B, Cabell C H, Naik S U, Klassen P and Panes J (2020) Efficacy and Safety of Etrasimod in a Phase 2 Randomized Trial of Patients With Ulcerative Colitis. Gastroenterology 158:550-561) and is currently under Phase 3 development for ulcerative colitis and Phase 2/3 development for Crohn's disease.

Described herein is the first clinical trial for an S1P modulator in patients with the dermatological indication atopic dermatitis. Lymphocyte reduction is an on-target effect of etrasimod. During the study, some investigators discontinued etrasimod due to lymphocyte levels meeting CTCAE grade 3 criteria. Although this presented challenges for the trial, it also provided an unexpected opportunity to evaluate the effects of interrupting and reinitiating treatment with etrasimod. An analysis of the trial results revealed that the patient cohort with dose interruption showed clinical rebound (or worsening of atopic dermatitis) following withdrawal and a resumption of clinical effect following a restart of treatment with etrasimod. Further, this observation was consistent with pharmacodynamic observations in that patient cohort. Importantly, none of these patients evidenced infection that would have warranted treatment interruption.

Provided is a method of treating or ameliorating at least one symptom or indication of an atopic dermatitis in an individual in need thereof, comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof.

Also provided is a method of treating or ameliorating at least one symptom or indication of atopic dermatitis in an individual in need thereof, comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof, wherein the Compound 1 or pharmaceutically acceptable salt thereof is administered an amount equivalent to more than 2 mg.

In some embodiments, a method of treating moderate to severe atopic dermatitis includes administering etrasimod, or a pharmaceutically acceptable salt thereof, to a patient in need thereof, identifying a threshold absolute lymphocyte count (ALC) in the patient, ceasing administration of the etrasimod, or a pharmaceutically acceptable salt thereof, for an interruption period of time, and after ceasing administration for the interruption period of time, continuing to administer the etrasimod, or a pharmaceutically acceptable salt thereof, for a continuation period of time, wherein the patient in need thereof experiences an improvement in moderate to severe atopic dermatitis. In some embodiments, the etrasimod, or a pharmaceutically acceptable salt thereof, is administered at a frequency of once a day. In some embodiments, the etrasimod, or a pharmaceutically acceptable salt thereof, is administered at a frequency of once a day and the improvement to moderate to severe atopic dermatitis includes an improvement in validated Investigator Global Assessment (vIGA) score. According to some embodiments, the etrasimod, or a pharmaceutically acceptable salt thereof, is administered at a frequency of once a day during the continuation period of time. In some embodiments, the etrasimod, or a pharmaceutically acceptable salt thereof, is administered in an amount of about 2 mg. In some embodiments, the threshold ALC is less than 500/mm3 or between 0.2×10e9 cells/L and 0.5×10e9 cells/L. In some embodiments, the threshold ALC is less than 200/mm3 or less than 0.2×10e9 cells/L. In some embodiments, the interruption period of time is at least one week. In some embodiments, the interruption period of time is between about one week and about four weeks. In some embodiments, the continuation period of time is at least one month. In some embodiments, the patient is diagnosed with moderate atopic dermatitis and has a vIGA score of 3. In some embodiments, the patient is diagnosed with severe atopic dermatitis and has a vIGA score of 4. According to some embodiments, the method further comprises the step of testing a circulating blood level of lymphocytes in the patient. In some embodiments, the method further comprises testing the circulating blood level of lymphocytes in the patient a subsequent time after the interruption period, identifying a threshold ALC in the patient, and, if the threshold is met, continuing to cease treatment. In some embodiments, the patient in need thereof exhibits a body surface area (BSA) of atopic dermatitis greater than or equal to 10%. In some embodiments, a method of treating moderate to severe atopic dermatitis includes administering etrasimod, or a pharmaceutically acceptable salt thereof, to a patient in need thereof, monitoring the patient for infections, ceasing administration of the etrasimod, or a pharmaceutically acceptable salt thereof, for an interruption period of time, and after ceasing administration for the interruption period of time, continuing to administer the etrasimod, or a pharmaceutically acceptable salt thereof, for a continuation period of time, wherein the patient in need thereof experiences an improvement in moderate to severe atopic dermatitis. According to some embodiments, the etrasimod, or a pharmaceutically acceptable salt thereof, is administered at a frequency of once a day during the continuation period of time. In some embodiments, the etrasimod, or a pharmaceutically acceptable salt thereof, is administered in an amount of about 2 mg. In some embodiments, the patient is diagnosed with moderate atopic dermatitis.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-2-(7-(4-cyclopentyl-3-(trifluoromethyl)benzyloxy)-1,2,3,4-tetrahydrocyclopenta[b]indol-3-yl)acetic acid including crystalline forms thereof.

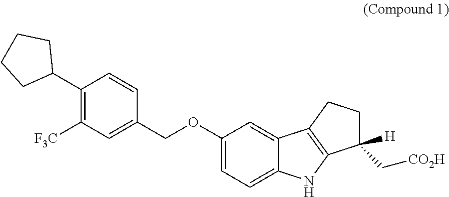 [SMILES: O=C(O)C[C@H]1CCc2c1[nH]c1ccc(OCc3ccc(C4CCCC4)c(C(F)(F)F)c3)cc21]

See PCT patent application, Serial No. PCT/US2009/004265 hereby incorporated by reference in its entirety. As a non-limiting example, Compound 1 may be present as an anhydrous, non-solvated crystalline form as described in WO 2010/011316 (incorporated by reference herein in its entirety). As another non-limiting example, an L-arginine salt of Compound 1 may be present as an anhydrous, non-solvated crystalline form as described in WO 2010/011316 and WO 2011/094008 (each of which is incorporated by reference herein in its entirety). As another non-limiting example, a calcium salt of Compound 1 may be present as a crystalline form as described in WO 2010/011316 (incorporated by reference herein in its entirety). Compound 1 is herein also described as “etrasimod”.

Compound 1, or a pharmaceutically acceptable salt thereof, is an orally administered, selective, synthetic sphingosine 1-phosphate (SIP) receptor 1, 4, 5 modulator. To date, Compound 1, or a pharmaceutically acceptable salt thereof, has been found to be safe and well-tolerated in clinical trials. Its safety and tolerability have been evaluated in Phase 1 studies with healthy adult subjects at single doses up to 5 mg and repeated doses up to 4 mg once daily (QD). In a Phase 2 dose-ranging study in UC patients, treatment with 2 mg QD for 12 weeks led to clinically meaningful and statistically significant endoscopic and symptomatic improvements versus placebo. Sustained beneficial effects of were observed in a subsequent open-label extension study.

MODERATE TO SEVERE ATOPIC DERMATITIS: As used herein, “moderate to severe atopic dermatitis,” in some embodiments means that one or more of the following features are present: (1) a minimum involvement of 10% body surface area (BSA); (2) regardless of BSA, individual lesions with moderate-to severe-features; involvement of highly visible areas or those important for function (e.g., neck, face, genitals, palms, and/or soles); and significantly impaired quality of life. According to some embodiments, a validated Investigator's Global Assessment (vIGA) of 3 is considered moderate atopic dermatitis, and a vIGA of 4 is considered severe atopic dermatitis. Those of skill in the art will also recognize and moderate to severe atopic dermatitis.

EXTRINSIC OR ALLERGIC: ATOPIC DERMATITIS: Extrinsic or allergic atopic dermatitis is atopic dermatitis with high total serum IgE levels and the presence of specific IgE for environmental and food allergens.

INTRINSIC OR NON-ALLERGIC ATOPIC DERMATITIS: Intrinsic or non-allergic atopic dermatitis is atopic dermatitis with normal total IgE values and the absence of specific IgE.

vIGA: As used herein, “vIGA” means validated Investigator's Global Assessment scale for AD, which is currently a five-point scale to measure disease severity. The vIGA score is selected using descriptors that best describe the overall appearance of skin lesions at a given time point using the following scoring: 0=clear (no inflammatory signs of AD); 1=almost clear (barely perceptible erythema and papulation); 2=mild (slight but definite erythema and papulation); 3=moderate (clearly perceptible erythema and papulation); and 4=severe (marked erythema papulation). The scale is further described at https://www.eczemacouncil.org/assets/docs/Validated-Investigator-Global-Assessment-Scale vIGA-AD 2017.pdf/, which is incorporated by reference in its entirety herein. In some embodiments, the standards for vIGA can be apparent to those of skill in the art.

EASI: As used herein, “EAST” means Eczema Area and Severity Index (EAST). EASI is an outcome measure for the clinical signs of AD. The current EASI is a composite index with scores ranging from 0 to 72. The EASI scoring assessment multiplies the percentage of the affected area in 4 specific disease characteristics (erythema, thickness [induration, papulation, edema], scratching [excoriation], and lichenification), which is assessed for severity by a physician on a scale of “0” (absent) through “3” (severe). The EASI Area Score can be documented for four regions of the body—Region 1: head and neck; Region 2: trunk (including genital area); Region 3: upper limbs; and Region 4: lower limbs (including buttocks), with the area of AD involvement assessed as a percentage by body area and converted to a score of 0 to 6. In each body region, the area is expressed as 0, 1 (1% to 9%), 2 (10% to 29%), 3 (30% to 49%), 4 (50% to 69%), 5 (70% to 89%), or 6 (90% to 100%). In some embodiments, the standards for EASI can be apparent to those of skill in the art.

EASI 75: As used herein, “EASI 75” means a 75% reduction of EASI from baseline.

EASI 50: As used herein, “EASI 50” means a 50% reduction of EASI from baseline.

EASI 90: As used herein, “EASI 90” means a 90% reduction of EASI from baseline.

PRURITUS NUMERIC RATING SCALE (NRS): As used herein, “pruritis numeric rating scale” or “NRS” refers to an assessment tool that patients use to report the intensity of their pruritus (itch). The scale for the NRS is from 0 to 10, with 0 being “no itch” and 10 being “the worst itch imaginable.” This scale is further described at http://www.pruritussymposium.de/numericalratingscale.html, which is incorporated by reference in its entirety. In some embodiments, the standards for NRS can be apparent to those of skill in the art.

ADMINISTERING: As used herein, “administering” means to provide a compound or other therapy, remedy, or treatment such that an individual internalizes a compound.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a sphingosine 1-phosphate subtype 1 (SIP₁) receptor-associated disorder. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention” such as prevention of a sphingosine 1-phosphate subtype 1 (SIP₁) receptor-associated disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

ADVERSE EVENT: As used herein, an “adverse event” is an untoward medical occurrence that is associated with treatment with Compound 1 or a pharmaceutically acceptable salt thereof.

INFECTION: As used herein, “infection” refers to the invasion and multiplication of microorganisms such as bacteria, viruses, and parasites that are not normally present within the body. An infection may cause no symptoms and be subclinical, or it may cause symptoms and be clinically apparent. An infection may remain localized, or it may spread through the blood or lymphatic vessels to become systemic (bodywide). Infections may include, but are not limited to urinary tract infection, common cold, diptheria, E. coli, giardiasis, HIV/AIDS, mononucleosis, influenza, lyme disease, measles, meningitis, mumps, pneumonia, salmonella infections, respiratory infections, shingles, herpes, tuberculosis, viral hepatitis, COVID-19, and those recognized by those of skill in the art.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL: As used herein, “individual” means a human. In some embodiments, an individual is referred to a “subject” or “patient.”

DOSE: As used herein, “dose” means a quantity of Compound 1, or a pharmaceutically acceptable salt thereof, given to the individual for treating or preventing the disease or disorder at one specific time.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient. The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

PHARMACEUTICAL COMPOSITION: As used here, “pharmaceutical composition” means a composition comprising at least one active ingredient, such as Compound 1; including but not limited to salts of Compound 1, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

AGONIST: As used herein, “agonist” means a moiety that interacts with and activates a G-protein-coupled receptor, such as the SIP₁ receptor, such as can thereby initiate a physiological or pharmacological response characteristic of that receptor. For example, an agonist activates an intracellular response upon binding to the receptor, or enhances GTP binding to a membrane. In certain embodiments, an agonist of the invention is an SIP₁ receptor agonist that is capable of facilitating sustained SIP₁ receptor internalization (see e.g., Matloubian et al., Nature, 427, 355, 2004).

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

In some embodiments, the Compound 1, or a pharmaceutically acceptable salt thereof, is administered orally.

In some embodiments, the Compound 1, or a pharmaceutically acceptable salt thereof, is formulated as a capsule or tablet suitable for oral administration.

In some embodiments, the Compound 1, or a pharmaceutically acceptable salt thereof, is selected from: Compound 1; a calcium salt of Compound 1; and an L-arginine salt of Compound 1. In some embodiments, the Compound 1, or a pharmaceutically acceptable salt thereof, is an L-arginine salt of Compound 1. In some embodiments, the Compound 1, or a pharmaceutically acceptable salt, thereof, is an anhydrous, non-solvated crystalline form of an L-arginine salt of Compound 1. In some embodiments, the Compound 1, or a pharmaceutically acceptable salt thereof, is an anhydrous, non-solvated crystalline form of Compound 1.

Also provided are pharmaceutical compositions comprising a standard dose of Compound 1, or a pharmaceutically acceptable salt thereof, and, optionally, one or more pharmaceutically acceptable carriers. Also provided are pharmaceutical compositions comprising Compound 1, or a pharmaceutically acceptable salt thereof, optionally, one or more pharmaceutically acceptable carriers. The carrier(s) must be “acceptable” in the sense of being compatible with the other ingredients of the formulation and not overly deleterious to the recipient thereof.

In some embodiments, Compound 1, or a pharmaceutically acceptable salt thereof, is administered as a raw or pure chemical, for example as a powder in capsule formulation.

In some embodiments, Compound 1, or a pharmaceutically acceptable salt thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

In some embodiments, the pharmaceutical dosage form is administered once daily to the individual.

In some embodiments, the individual is administered an amount equivalent to about 0.5 to about 5.0 mg of Compound 1. In some embodiments, the individual is administered an amount equivalent to 1 mg of Compound 1. In some embodiments, the individual is administered an amount equivalent to 2 mg of Compound 1. In some embodiments, a patient is administered an amount equivalent to more than 2 mg of Compound 1. In some embodiments, a patient is administered an amount equivalent to more than 2.5 mg of Compound 1. In some embodiments, the individual is administered an amount equivalent to 2.25 mg of Compound 1. In some embodiments, the individual is administered an amount equivalent to 2.5 mg of Compound 1. In some embodiments, the individual is administered an amount equivalent to 2.75 mg of Compound 1. In some embodiments, the individual is administered an amount equivalent to 3 mg of Compound 1. In some embodiments, a patient is administered an amount equivalent to, or of about, 2.1, 2.2, 2.25, 2.3, 2.4, 2.5, 2.6, 2.7, 2.75, 2.8. 2.9, or 3.0 mg of Compound 1. In some embodiments, a patient is administered more than, or more than about, 2 mg of Compound 1 for greater efficacy than may be achieved with 2 mg of Compound 1. In some embodiments, a patient is administered more than, or more than about, 2 mg of Compound 1 to maximize EASI change from baseline at week 12.

In some embodiments, the individual is administered an amount equivalent to 1 mg of Compound 1 for a first time period and subsequently an amount equivalent to 2 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is indefinite, e.g., chronic administration.

In some embodiments, the standard dose is administered without titration. In some embodiments, the standard dose is administered without titration; and the individual does not experience a severe related adverse event. In some embodiments, the standard dose is administered without requiring titration to avoid first-dose effect seen with other S1P receptor modulators.

In some embodiments, the dosage form is administered under fasted conditions. In some embodiments, the dosage form is administered under fed conditions.

In some embodiments, the method is non-gender specific.

In some embodiments, Compound 1, or a pharmaceutically acceptable salt thereof, is administered in combination with a second therapeutic agent or therapy, wherein the second therapeutic agent or therapy is selected from an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management.

In some embodiments, Compound 1, or a pharmaceutically acceptable salt thereof, was previously administered at least one therapeutic agent or therapy, wherein the therapeutic agent or therapy is selected from an IL-1beta inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a glucocorticoid, a proton pump inhibitor, a NSAID, allergen removal and diet management.

In some embodiments, the individual is, or was, treated with an IL-1beta inhibitor. In some embodiments, the IL-1beta inhibitor is anakinra, rilonacept, or canakinumab.

In some embodiments, the individual is, or was, treated with an IL-5 inhibitor. In some embodiments, the IL-5 inhibitor is benralizumab, mepolizumab or reslizumab.

In some embodiments, the individual is treated with an IL-9 inhibitor.

In some embodiments, the individual is, or was, treated with an IL-13 inhibitor. In some embodiments, the IL-13 inhibitor is lebrikizumab, RPC4046, or tralokinumab.

In some embodiments, the individual is, or was, treated with an IL-17 inhibitor. In some embodiments, the IL-17 inhibitor is ixekizumab or brodalumab.

In some embodiments, the individual is, or was, treated with an IL-25 inhibitor.

In some embodiments, the individual is, or was, treated with a TNFα inhibitor. In some embodiments, the TNFα inhibitor is SIMPONI® (golimumab), REMICADE® (infliximab), HUMIRA® (adalimumab), or CIMZIA® (certolizumab pegol).

In some embodiments, the individual is, or was, treated with an eotaxin-3 inhibitor. In some embodiments, the individual is, or was, treated with an IgE inhibitor. In some embodiments, the IgE inhibitor is omalizumab.

In some embodiments, the individual is, or was, treated with a prostaglandin D2 inhibitor. In some embodiments, the individual is, or was, treated with an immunosuppressant. In some embodiments, the immunosuppressant is AZASAN® (azathioprine), IMURAN® (azathioprine), GENGRAF® (cyclosporine), NEORAL® (cyclosporine), or SANDIMMUNE® (cyclosporine). Immunosuppressants also may be referred to as immunosuppressives or immunosuppressive agents.

In some embodiments, the individual is, or was, treated with a glucocorticoid. In some embodiments, the glucocorticoid is UCERIS® (budesonide); DELTASONE® (prednisone), MEDROL® (methylprednisolone), or hydrocortisone. Glucocorticosteroids also may be referred to as glucocorticoid or corticosteroids.

In some embodiments, the individual is, or was, treated with a NSAID. In some embodiments, the NSAID is aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In some embodiments, the individual is, or was treated with DUPIXENT® (dupilumab).

In some embodiments, the treatment is for inducing clinical remission. In some embodiments, the treatment is for maintaining clinical remission. In some embodiments, the treatment is for inducing and maintaining clinical remission.

In some embodiments, the treatment is for inducing clinical response. In some embodiments, the treatment is for maintaining clinical response. In some embodiments, the treatment is for inducing and maintaining clinical response.

Also provided is a method of treating an individual with atopic dermatitis, comprising administering to the individual in need thereof a therapeutically effective amount of etrasimod, or a pharmaceutically acceptable salt thereof. In some embodiments, the atopic dermatitis is moderate to severe atopic dermatitis. In some embodiments, the atopic dermatitis is moderate atopic dermatitis. In some embodiments, the atopic dermatitis is severe atopic dermatitis. In some embodiments, the atopic dermatitis is chronic atopic dermatitis. In some embodiments, the individual with atopic dermatitis is inadequately controlled by or intolerant to topical therapy. In some embodiments, the individual with atopic dermatitis is inadequately controlled by or intolerant to biologics. In some embodiments, the individual with atopic dermatitis is inadequately controlled by or intolerant to dupilumab.

Also provided is a method of treating an individual with pruritis, comprising administering to the individual in need thereof a therapeutically effective amount of etrasimod, or a pharmaceutically acceptable salt thereof. In some embodiments, the pruritis is moderate to severe pruritis. In some embodiments, the pruritis is moderate pruritis. In some embodiments, the pruritis is severe pruritis.

In some embodiments, the methods described herein achieve a therapeutic effect. In some embodiments, the method is therapeutically effective to improve EASI from baseline, to achieve EASI-75, to achieve a vIGA 0 to 1 (on a 5-point scale) score, to reduce a patient's peak pruritus NRS, to reduce the percent BSA AD involvement from baseline, to achieve EASI-50, defined as a ≥50% reduction of EASI from baseline, or to achieve EASI-90 (defined as a ≥90% reduction of EASI from baseline).

In some embodiments, the therapeutic effect is observed in, or in about, four weeks, five weeks, six weeks, seven weeks, eight weeks, ten weeks, twelve weeks, or sixteen weeks. In some embodiments, the therapeutic effect is observed in, or in about, four weeks, five weeks, six weeks, seven weeks, eight weeks, ten weeks, twelve weeks, or sixteen weeks of initiating treatment. In some embodiments, the therapeutic effect is observed in, or in about, four weeks, five weeks, six weeks, seven weeks, eight weeks, ten weeks, twelve weeks, or sixteen weeks of reinitiating treatment. In some embodiments, the method is effective to treat pruritis in a patient diagnosed with AD in, or in about, four weeks. In some embodiments, the method is effective to demonstrate a statistically significant improvement in EASI from baseline in four weeks.

In some embodiments, the improvement in moderate to severe atopic dermatitis comprises an improvement in a patient-reported outcome assessed using the Dermatology Life Quality Index (DLQI), Patient Oriented Eczema Measure (POEM), or Patient Global Assessment (PGA).

In some embodiments, the improvement to moderate to severe atopic dermatitis comprises an improvement in pruritus.

According to some embodiments, a method of treatment includes monitoring the circulating blood lymphocyte levels of a patient suffering from atopic dermatitis and adjusting the treatment if the absolute lymphocyte count (ALC) levels are below an identified threshold. In some embodiments, the threshold is ALC less than, or less than about, 500/mm³. In some embodiments, the threshold is ALC less than. or less than about, 500/mm³, 450/mm³, 400/mm³, 350/mm³, 300/mm³, 250/mm³, or 200/mm³. In some embodiments, the ALC is between, or between about, 0.2×10e9 cells/L and 0.5×10e9 cells/L. In some embodiments, the ALC is less than, or less than about, 0.5×10e9 cells/L, 0.45×10e9 cells/L, 0.4×10e9 cells/L, 0.35×10e9 cells/L, 0.3×10e9 cells/L, 0.25×10e9 cells/L, or 0.2×10e9 cells/L. In some embodiments, the threshold is an ALC that meets grade 3 criteria as defined by the Common Terminology Criteria for Adverse Events (CTCAE), as described at https://ctep.cancer.gov/protocolDevelopment/electronic_applications/docs/CTCAE_v5_Quick_Reference_8.5×11.pdf (lymphocyte count decreased), which is herein incorporated by reference in its entirety. In some embodiments, the threshold is an ALC that meets grade 4 criteria as defined by the CTCAE. In some embodiments, the threshold is ALC less than 200/mm³. In some embodiments, the threshold is ALC less than, or less than about, 0.2×10e9 cells/L.

In some embodiments, the patient is monitored or screened for infection during treatment. An infection may be identified by diagnosis by a physician based on physical signs and symptoms. In some embodiments, a patient may be tested for certain infections based on known diagnostic tests.

In some embodiments, if the ALC levels are below the threshold level, an interruption period of time is initiated and treatment is ceased (the patient does not receive etrasimod during the interruption period). In some embodiments, the interruption period is between one day and two months. In some embodiments, the interruption period is between one week and six weeks. In some embodiments, the interruption period is between one week and four weeks. In some embodiments, the interruption period is between one week and three weeks. In some embodiments, the interruption period is one day, two days, three days, four days, five days, six days, seven days (one week), ten days, two weeks, three weeks, four weeks, five weeks, six weeks, seven weeks, eight weeks, nine weeks, ten weeks, eleven weeks, twelve weeks, or thirteen weeks. After an interruption period, the serum levels of ALC in the patient can be retested, and if the ALC levels are above the threshold, then the patient can continue to receive etrasimod in a continuation period of time. After an interruption period, the serum levels of ALC in the patient can be retested, and if the ALC levels are below the threshold level, then the patient can enter a subsequent interruption period. In some embodiments, the subsequent interruption period is between one day and two months. In some embodiments, the subsequent interruption period is between one week and six weeks. In some embodiments, the subsequent interruption period is between one week and four weeks. In some embodiments, the subsequent interruption period is between one week and three weeks. In some embodiments, the subsequent interruption period is one day, two days, three days, four days, five days, six days, seven days (one week), ten days, two weeks, three weeks, four weeks, five weeks, six weeks, seven weeks, eight weeks, nine weeks, ten weeks, eleven weeks, twelve weeks, or thirteen weeks. After a subsequent interruption period, the serum levels of ALC in the patient can be retested, and if the ALC level are above the threshold, then the patient can continue to receive etrasimod in a continuation period of time. After an interruption period, the serum levels of ALC in the patient can be retested, and if the ALC levels are below the threshold, then the patient can enter another subsequent interruption period.

In some embodiments, if an infection is identified, an interruption period of time is initiated and treatment is ceased (the patient does not receive etrasimod during the interruption period). In some embodiments, the interruption period is between one day and two months. In some embodiments, the interruption period is between one week and six weeks. In some embodiments, the interruption period is between one week and four weeks. In some embodiments, the interruption period is between one week and three weeks In some embodiments, the interruption period is one day, two days, three days, four days, five days, six days, seven days (one week), ten days, two weeks, three weeks, four weeks, five weeks, six weeks, seven weeks, eight weeks, nine weeks, ten weeks, eleven weeks, twelve weeks, or thirteen weeks. After an interruption period, the patient can be screened again for an infection and if an infection is not identified, then the patient can continue to receive etrasimod in a continuation period of time. After an interruption period, the patient can be screened again for an infection and if an infection is identified, then the patient can enter a subsequent interruption period. In some embodiments, the subsequent interruption period is between one day and two months. In some embodiments, the subsequent interruption period is between one week and six weeks. In some embodiments, the subsequent interruption period is between one week and four weeks. In some embodiments, the subsequent interruption period is between one week and three weeks. In some embodiments, the subsequent interruption period is one day, two days, three days, four days, five days, six days, seven days (one week), ten days, two weeks, three weeks, four weeks, five weeks, six weeks, seven weeks, eight weeks, nine weeks, ten weeks, eleven weeks, twelve weeks, or thirteen weeks. After a subsequent interruption period, the patient can be screened again for an infection and if an infection is not identified, then the patient can continue to receive etrasimod in a continuation period of time. After a subsequent interruption period, the patient can be screened again for an infection and if an infection is identified, then the patient can enter another subsequent interruption period.

During a continuation period of time, a patient reinitiates treatment with etrasimod, or a pharmaceutically acceptable salt thereof. In some embodiments, the continuation period is between one day and two months. In some embodiments, the continuation period is between one week and six weeks. In some embodiments, the continuation period is between one month and two years. In some embodiments, the continuation period is until the AD substantially resolves (e.g., a vIGA score of 0 or 1). In some embodiments, the continuation period is between one week and one year. In some embodiments, the continuation period is between one week and four weeks. In some embodiments, the continuation period is between one week and two weeks. In some embodiments, the continuation period is at least one week, one month, two months, three months, four months, five months, six months, seven months, ten months, eleven months, twelve months, thirteen months, fifteen months, twenty months or twenty-four months. In some embodiments, during the continuation period, the patient can be administered etrasimod or a pharmaceutically acceptable salt thereof at a frequency of once a day. In some embodiments, during the continuation period, the patient can be administered etrasimod or a pharmaceutically acceptable salt thereof in an amount equivalent to 1 mg or 2 mg or 3 mg of etrasimod a day. In some embodiments, during the continuation period, the patient can be administered etrasimod or a pharmaceutically acceptable salt thereof in an amount equivalent to 2 mg of etrasimod a day.

In some embodiments, the methods described herein achieve a therapeutic effect during the continuation period of time. In some embodiments, the method is therapeutically effective to improve EASI from baseline, to achieve EASI-75, to achieve a vIGA 0 to 1 (on a 5-point scale) score, to reduce a patient's vIGA by at least 1 point, to reduce a patient's peak pruritus NRS, to reduce the percent BSA AD involvement from baseline, to achieve EASI-50, defined as a ≥50% reduction of EASI from baseline, or to achieve EASI-90, defined as a ≥90% reduction of EASI from baseline during the continuation period of time.

In some embodiments, after treatment is reinitiated, the patient experiences an improvement in EASI score that is equal to or within 25% of the EASI score if the patient had not experienced an interruption period. In some embodiments, after treatment is reinitiated, the patient experiences an improvement in vIGA score that is equal to or within 25% of the vIGA score if the patient had not experienced an interruption period. In some embodiments, after treatment is reinitiated, the patient experiences an improvement in pruritis that is equal to or within 25% of the treatment effect for pruritis if the patient had not experienced an interruption period.

In some embodiments, once treatment reinitiates, a rapid recapture of therapeutic effect is achieved. In some embodiments, when a rapid recapture occurs, the patient can exhibit a therapeutic effect equal to or better than the patient's measurement of the effect immediately before the interruption period began. In some embodiments, the patient achieves a vIGA score equal to or better than the patient's measurement of the effect immediately before the interruption period began. In some embodiments, the patient achieves an EASI equal to or better than the patient's measurement of the effect immediately before the interruption period began. In some embodiments, the patient achieves an NRC equal to or better than the patient's measurement of the effect immediately before the interruption period began. According to some embodiments, the recapture of therapeutic effect occurs within one day, two days, three days, four days, five days, six days, seven days (one week), ten days, two weeks, three weeks, or one month of the start of the continuation period.

In some embodiments, a lymphocyte level in a patient is used as a biomarker. In some embodiments, ALC in a patient is used as a biomarker. In some embodiments, the biomarker is used to determine dose and/or frequency of administration for the patient. In some embodiments, the biomarker is used to determine whether treatment should be interrupted. In some embodiments, the biomarker is used to reinitiate treatment following interruption. In some embodiments, the biomarker is used to expedite reinitiation of treatment following interruption. In some embodiments, the biomarker is a lymphocyte count that meets grade 3 criteria as defined CTCAE. In some embodiments, the biomarker is a lymphocyte count that meets grade 4 criteria as defined CTCAE. In some embodiments, the biomarker is an ALC that meets grade 3 criteria as defined CTCAE. In some embodiments, the biomarker is an ALC that meets grade 4 criteria as defined CTCAE.

Further embodiments include the embodiments disclosed in the following Examples, which is not to be construed as limiting in any way.

### EXAMPLES

### Example 1

Formulations composed of immediate-release tablets containing an L-arginine salt of Compound 1 were prepared as shown in Table 1.

TABLE 1

| Tablet Strength | 1 mg | 2 mg | Placebo |
|---|---|---|---|
| L-Arg Salt of Compound 1 | 1.381 | 2.762 | 0 |
| Mannitol Pearlitol ® 100SD | 54.119 | 52.738 | 55.5 |
| Microcrystalline cellulose - | 40 | 40 | 40 |
| Sodium Starch Glycolate - | 4 | 4 | 4 |
| Magnesium Stearate | 0.5 | 0.5 | 0.5 |
| Opadry ® II Blue | 4 | 4 | 4 |
| Total tablet target weight | 104 | 104 | 104 |

### Example 2

A Phase 2, multicenter, randomized, double-blind, placebo-controlled study was conducted to assess the safety and efficacy of etrasimod in patients with moderate to severe atopic dermatitis. The primary endpoint measured was percent change in Eczema Area and Severity Index (EAST) from baseline to week 12, followed by a 4-week follow-up observation period. Secondary endpoints include the proportion of participants achieving EASI-75, proportion of participants with a validated Investigator Global Assessment (vIGA) 0 to 1, and percent change in peak pruritis. The trial enrolled approximately 140 patients and was conducted in study sites across the United States, Canada and Australia. An open-label extension of the trial is ongoing.

The study included multiple periods: (i) up to a 4-week screening period (to determine subject eligibility); (ii) a 12-week double-blind treatment period with a 4-week safety follow-up period; and (iii) a 52-week open-label extension period followed by a 4-week safety follow-up period. Subjects with chronic AD for at least one year (as defined by Hanifin and Rajka criteria (Hanifin 1980)) despite optimized skin care (e.g., use of emollients, avoidance of irritants) whose disease is not adequately controlled with topical therapies, or for whom those therapies are not advisable, were equally randomized (1:1:1 ratio) to receive etrasimod (1 or 2 mg) or placebo. Etrasimod 1 or 2 mg tablets or matching placebo tablets were taken orally once daily during the double-blind treatment period. Etrasimod 2 mg tablets are taken orally once daily during the open-label extension period. There is no reference therapy (no placebo) during the open-label extension period.

Inclusion Criteria: Subjects must meet ALL of the following inclusion criteria to be eligible for enrollment into the study.
1. Men or women between ≥18 and ≤70 years of age at the time of informed consent.
2. Chronic AD, defined by Hanifin and Rajka criteria (Hanifin 1980), that has been present for at least one year prior to the screening visit.
3. Eczema Area and Severity Index (EAST)≥12 at the screening visit and ≥16 at the baseline visit.
4. vIGA score≥3 (on the 0 to 4 vIGA scale, in which 3=moderate and 4=severe) involvement at the screening and baseline visits.
5. Body surface area (BSA)≥10% of AD involvement at the screening and baseline visits.
6. Recent history (within 6 months prior to the screening visit) of inadequate response to treatment with topical medications, or when topical treatments are otherwise medically inadvisable (e.g., because of important side effects or safety risks), which can be documented by medical records or by the history provided to the investigator by the subject despite optimized skin care (.e., avoidance of irritants, use of emollients).
7. Willing to apply a dose of topical emollient/moisturizer at least once daily for ≥1 week prior to the baseline visit and willing to continue daily application over the course of the study without change (i.e., type, frequency, application).
8. Willing and able to comply with all clinic visits and study-related procedures and understand and complete study-related questionnaires.
9. Provide signed informed consent prior to conducting any procedures.

### Exclusion Criteria

1. Treatment with the following medications within the last 4 weeks before screening or during Screening: a) systemic immunosuppressive/immunomodulating drugs (e.g., methotrexate, cyclosporine), including oral Janus kinase (JAK) inhibitors for any indication, and off-label use; b) systemic glucocorticoids (excludes topical, inhaled, or intranasal delivery that are considered topical for any reason); c) immunoglobulin or blood products.
2. Treatment with the following medications within 1 week of randomization: a) topical corticosteroids, b) topical calcineurin inhibitors, c) topical crisaborole, d) topical JAK inhibitors (if not investigational).
3. Phototherapy for AD or artificial tanning (beds, booths, or lamps) within 4 weeks prior to screening or during screening.
4. Presence of skin comorbidities that will interfere with study assessments of the underlying disease.
Any cell-depleting agents, including but not limited to rituximab, within 6 months prior to the baseline visit or until lymphocyte count returns to normal, whichever is longer.
6. Use of dupilumab within 8 weeks prior to screening or during screening.
7. Use of any biological agents other than dupilumab (e.g., adalimumab, ustekinumab, secukinumab) regardless of indication, within 5 half-lives (if known) or 16 weeks prior to screening, whichever is longer, or during screening.
8. Received any investigational agent, including non-biologic agents and topical agents to treat AD, within 4 weeks or 5 half-lives (whichever is longer) prior to screening.
9. Initiation, during the screening period, of treatment of AD with prescription emollients or moisturizers containing additives such as ceramide, hyaluronic acid, urea, or filaggrin degradation products. (Note: Subjects may continue using stable doses of such emollients or moisturizers if they have been used for at least 8 weeks prior to the screening visit.)
10. Moderate or strong inducers/inhibitors of cytochrome P450 (CYP)2C8 and CYP2C9 (e.g., clopidogrel, gemfibrozil, fluconazole, and carbamazepine) during screening (this includes St. John's Wort). Exclusionary criteria related to medical history
11. Known active bacterial, viral, fungal, mycobacterial infection, or other infection (including tuberculosis [TB] or atypical mycobacterial disease) or any major episode of infection that required hospitalization or treatment with intravenous antibiotics within 4 weeks prior to screening or during screening, or oral antibiotics within 2 weeks prior to screening or during screening. Superficial fungal infection of the nail bed is allowed.
12. Have any of the following conditions or risk factors: a. Primary or secondary immunodeficiency syndromes (e.g., hereditary immunodeficiency syndrome, acquired immunodeficiency syndrome, drug-induced immune deficiency); b. History of organ transplant (except corneal transplant); c. History of an opportunistic infection (e.g., Pneumocystis jirovecii pneumonia, cryptococcal meningitis, progressive multifocal leukoencephalopathy [PML]); d. History of disseminated herpes simplex or disseminated herpes zoster, or any episode of herpes zoster; e. Test positive for human immunodeficiency virus, hepatitis B (positive for Hepatitis B surface antigen [HBsAg]), or active hepatitis C (with detectable viral load) at screening; f. Test positive for active TB with interferon-gamma release assay (IGRA; e.g., QuantiFERON-TB Gold In-Tube, T-SPOT TB) during screening. Those with known history of active TB or with positive test for latent TB at Screening may be included if 1 of the following are documented: Subjects with latent TB, who have been ruled out for active TB according to local country guidelines, (e.g., chest X-ray), have completed an appropriate course of TB prophylaxis treatment, and have not had recent close contact with a person with active TB are eligible to enroll in the study. It is the responsibility of the investigator to verify the adequacy of previous TB treatment and provide appropriate documentation; subjects diagnosed with latent TB at screening, ruled out for active TB, and who received at least 4 weeks of an appropriate TB prophylaxis regimen may be rescreened for enrollment.
13. Received any live or live-attenuated vaccines≤4 weeks prior to baseline.
14. History of malignancy of any organ system (other than localized squamous cell or basal cell carcinoma of the skin that have been excised or resolved), treated or untreated, within the past 5 years.
15. Any history of the following, unless treated with an implanted pacemaker or an implanted cardioverter-defibrillator with pacing: a) history or presence of symptomatic bradycardia; b) history of sick sinus syndrome or neurocardiogenic syncope; c) second or third-degree atrioventricular (AV) block; d) periods of asystole>3 seconds.
16. Have initiated or had a change in dose of Class I-IV cardiac antiarrhythmic therapy within 1 week prior to starting study treatment or anticipate starting antiarrhythmic therapy within 1 week after starting study treatment.
17. Have any of the following conditions that may affect cardiovascular function: a) experienced a myocardial infarction, unstable angina, decompensated heart failure requiring hospitalization, or Class III/IV heart failure≤2 months prior to screening, or during screening; b) history of recurrent syncope, or low heart rate (HR) and blood pressure (BP) at screening and Day 1 pre-dose (taken in the seated position): HR<50 bpm, systolic BP<90 mm Hg, and diastolic BP<55 mm Hg; c) screening and pre-randomization 12-lead electrocardiogram (ECG) showing clinically significant abnormalities with a PR interval≥220 ms, Fridericia's corrected QT interval (QTcF)≥450 ms (men) or QTcF≥470 ms (women).
18. A history of or active diabetic retinopathy, uveitis, retinitis pigmentosum or macular edema. Any recent intraocular surgery within one year of screening.
19. Active severe pulmonary disease (e.g., chronic obstructive pulmonary disease, pulmonary fibrosis) or chronic pulmonary disease requiring intravenous corticosteroid treatment or hospitalization≤12 months prior to screening or during screening. Exclusionary criteria related to test or laboratory results (performed by central laboratory) Note: A confirmed result means there have been two consecutive assessments showing a consistent abnormal, clinically relevant result.
20. Confirmed absolute lymphocyte count<0.8×109 cells/L at screening.
21. Confirmed estimated glomerular filtration rate<30 mL/min/1.73 m2 by the Chronic Kidney Disease Epidemiology Collaboration equation at screening.

22. Any of the following liver function test values during Screening:⋅Aspartate aminotransferase (AST)>2× the upper limit of normal (ULN)⋅Alanine aminotransferase (ALT)>2×ULN⋅Direct (conjugated) bilirubin that is >1.5×ULN, unless in the context of Gilbert's syndrome.

### General Exclusionary Criteria

23. Lactating female who is breastfeeding.
24. Females must be nonpregnant, evidenced by a negative serum beta-human chorionic gonadotropin (β-hCG) pregnancy test at screening and urine dipstick pregnancy test at Day 1, and meet either a or b of the following criteria and males must meet criterion c to qualify for the study:
a. A female who is not of childbearing potential must meet one of the following: postmenopausal, defined as no menses for 12 months without an alternative medical cause; permanent sterilization procedure, such as hysterectomy, bilateral salpingectomy, or bilateral oophorectomy;
b. A female who is of childbearing potential must agree to using a highly effective contraception method during treatment and for 4 weeks following treatment that can achieve a failure rate of less than 1% per year when used consistently and correctly. The following are considered highly effective birth control methods: Combined (estrogen and progestogen containing) hormonal contraception associated with inhibition of ovulation, which may be oral, intravaginal, or transdermal; progestogen-only hormonal contraception associated with inhibition of ovulation, which may be oral, injected, or implanted; intrauterine device; intrauterine hormone-releasing system; bilateral tubal occlusion; vasectomized partner; sexual abstinence (complete sexual abstinence defined as refraining from heterosexual intercourse for the entire period of risk associated with study treatments). The reliability of sexual abstinence needs to be evaluated in relation to the duration of the clinical study and the preferred and usual lifestyle of the subject. Periodic abstinence (calendar, symptothermal, post-ovulation methods) is not acceptable;
c. A male must agree to using condoms during treatment and for 4 weeks following treatment.
25. Any acute illness or medical condition including cognitive impairment and alcohol/drug abuse/dependence, or signs/symptoms suspicious for a serious disease that, in the investigator's opinion, could put the subject at increased risk for safety event(s), could interfere with participation in the study according to the study protocol, or with the ability of the subject to cooperate and comply with the study procedures.

Efficacy was assessed by changes in AD severity using EASI, vIGA, SCORing Atopic Dermatitis (SCORAD), and BSA. Symptoms of itch were assessed using the pruritus numeric rating scale (NRS). Patient-reported outcomes will also be assessed using the Dermatology Life Quality Index (DLQI), Patient Oriented Eczema Measure (POEM), and Patient Global Assessment (PGA). Efficacy endpoints investigated included (i) percent change in EASI from baseline to Week 12, (ii) proportion of subjects achieving EASI-75 (defined as a 75% reduction of EASI from baseline to Week 12), (iii) proportion of subjects with a vIGA 0 to 1 (on a 5-point scale) score and a reduction from baseline of ≥2 points at Week 12, (iv) percent change in peak pruritus NRS from an itch daily diary from baseline to Week 12, (v) proportion of subjects with improvement (reduction) in peak pruritus (vi) NRS≥3 from an itch daily diary from baseline to Week 12, (vii) change and percentage change in percent BSA AD involvement from baseline to Week 12, (viii) proportion of subjects achieving EASI-50 (defined as a ≥50% reduction of EASI from baseline to Week 12), and (ix) proportion of subjects achieving EASI-90 (defined as a ≥90% reduction of EASI from baseline to week 12).

During the double-blind treatment period, etrasimod plasma concentrations and a complete blood count (CBC) including lymphocyte count were assessed on day 1 and before dosing at subsequent study visits. Study visits occurred at baseline, week 1, week 2, week 4, week 6, week 8, week 12, at the end of treatment, and at the week 16 follow-up visit. The absolute lymphocyte count (ALC) was included as part of the assessment.

### Results

Results for the double-blind treatment period are as follows. 140 patients were randomized into three treatment groups: 1 mg etrasimod (n=47), 2 mg etrasimod (n=47), and placebo (n=46). Approximately 83% of patients enrolled in the study were diagnosed with moderate atopic dermatitis (vIGA of 3) at baseline and the remainder were diagnosed with severe atopic dermatitis (vIGA of 4) at baseline.

Treatment with 2 mg etrasimod improved patient-reported outcomes and clinician-assessed outcomes versus placebo over 12 weeks. Treatment with 2 mg etrasimod resulted in significantly greater proportions of patients achieving a vIGA score of 0 or 1, DLQI, and POEM at week 12 compared to placebo.

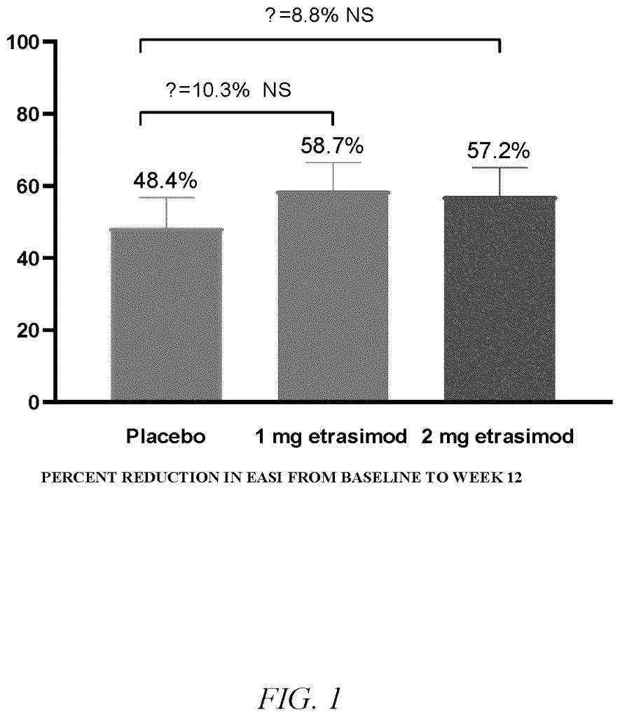

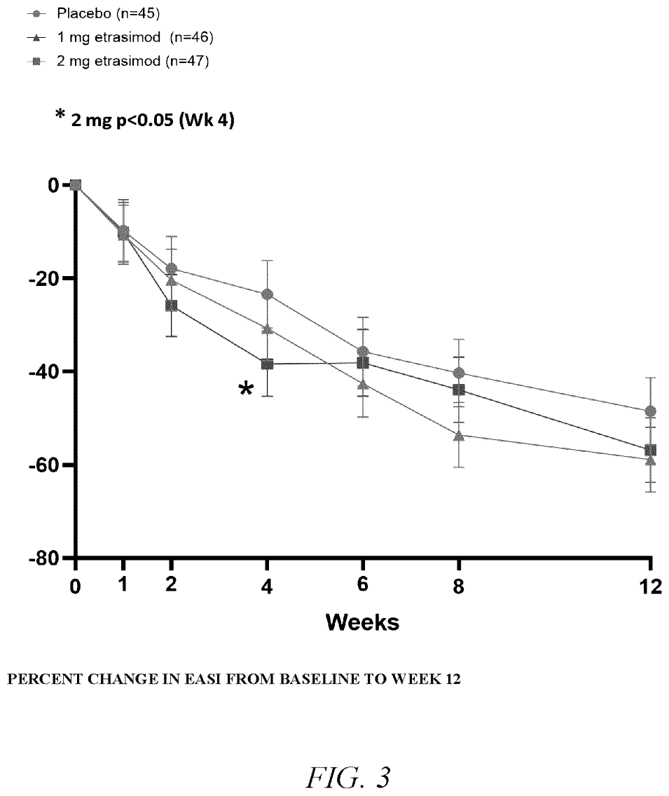

As shown in FIG. 1, patients receiving 1 mg of etrasimod showed a 58.7 percent reduction in EASI from baseline to week 12. Patients receiving 2 mg of etrasimod showed a 57.2% reduction in EASI from baseline to week 12. Patients receiving a placebo showed a 48.4% reduction in EASI from baseline to week 12. As such, EASI improved in all three groups through 12 weeks. However, as shown in FIG. 3, patients in the 2 mg dose group demonstrated a statistically significant improvement in EASI from baseline to week 4.

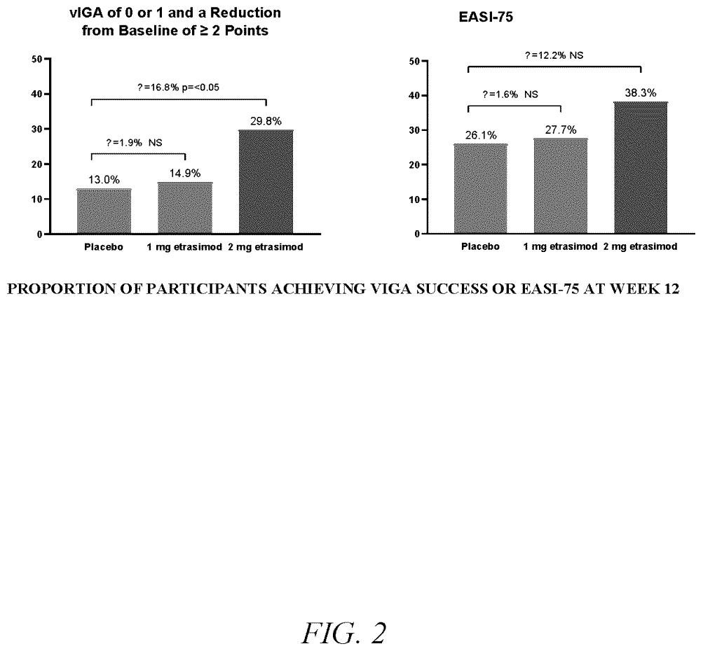

As shown in FIG. 2, 29.8% of 2 mg etrasimod patients achieved a vIGA of 0 or 1 and a reduction from baseline of greater than or equal to 2 points at week 12. 14.9% of 1 mg etrasimod patients achieved a vIGA of 0 or 1 and a reduction from baseline of greater than or equal to 2 points at week 12. 13.0% of placebo patients achieved a vIGA of 0 or 1 and a reduction from baseline of greater than or equal to 2 points at week 12. The 2 mg group achieved statistically significant success in this measure as compared to placebo, with a delta of 16.8% (p<0.05).

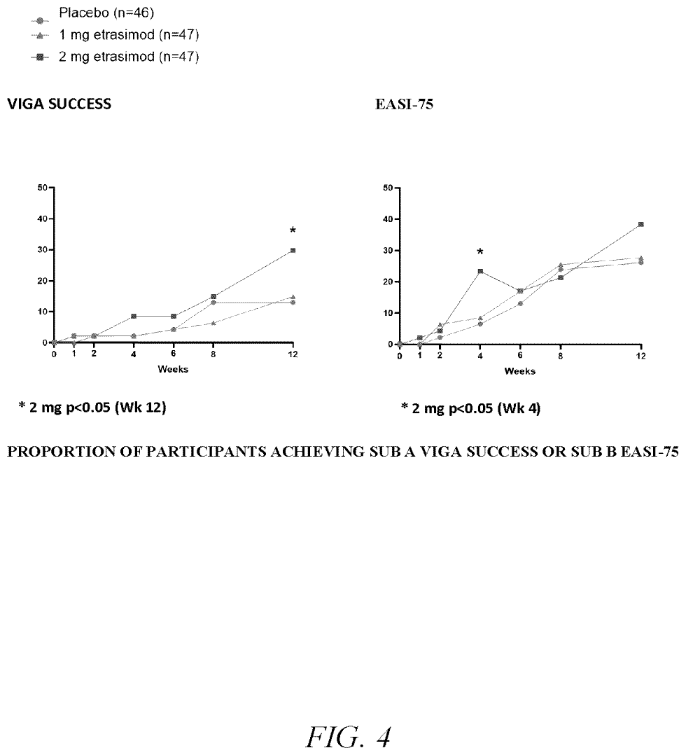

As shown in FIGS. 2 and 4, 38.3% of 2 mg etrasimod patients achieved an EASI-75. 27.7% of 1 mg etrasimod patients achieved an EASI-75. 26.1% of placebo patients achieved an EASI-75. A statistically significant improvement in the proportion of patients who achieved EASI-75 was observed at week 4 in 2 mg etrasimod dose group.

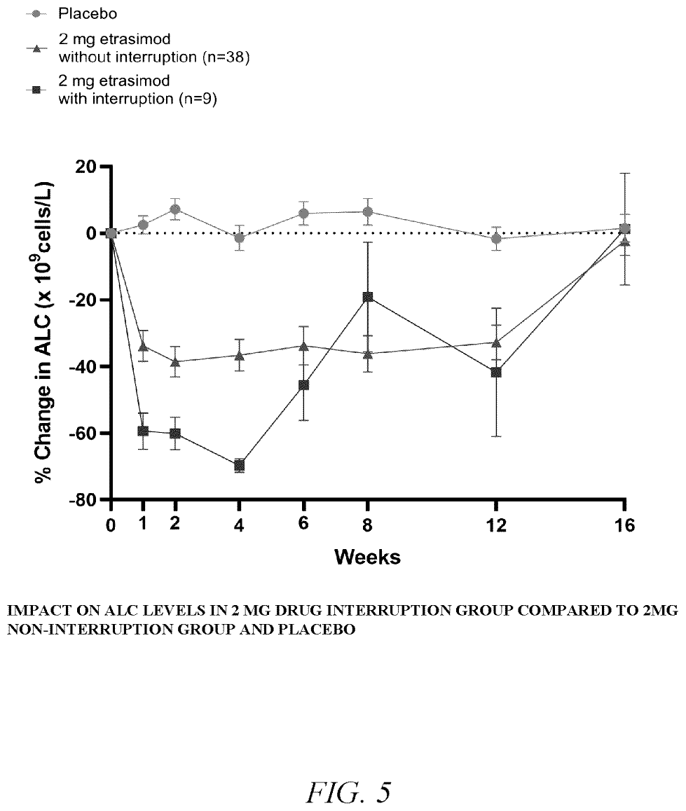

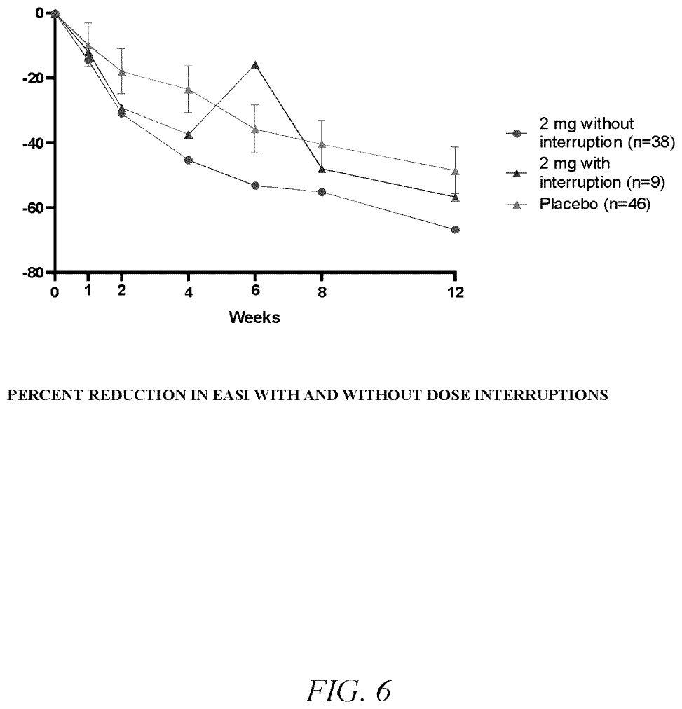

During the double-blind treatment period, nine patients in the 2 mg etrasimod group demonstrated an ALC measurement of Grade 3 (between 0.2×10e9 cells/L and 0.5×10e9 cells/L) between weeks 4 and 8. Treatment was withdrawn for these patients and reinitiated for a subset. The average length of drug interruption was 2 weeks, with the majority of patients continuing therapy at 8 weeks. Treatment response was recaptured once the etrasimod treatment was restarted. As shown in FIG. 5, there was a rapid rebound in lymphocyte count between weeks 4 and 8. The majority of patients resumed study drug by week 8 with and exhibited a subsequent decrease in lymphocyte count between weeks 8 and 12. As shown in FIG. 6, surprisingly, the EASI change from baseline for the patients who ceased treatment then reinitiated treatment improved rapidly after resumption, and their EASI continued to improve to week 12.

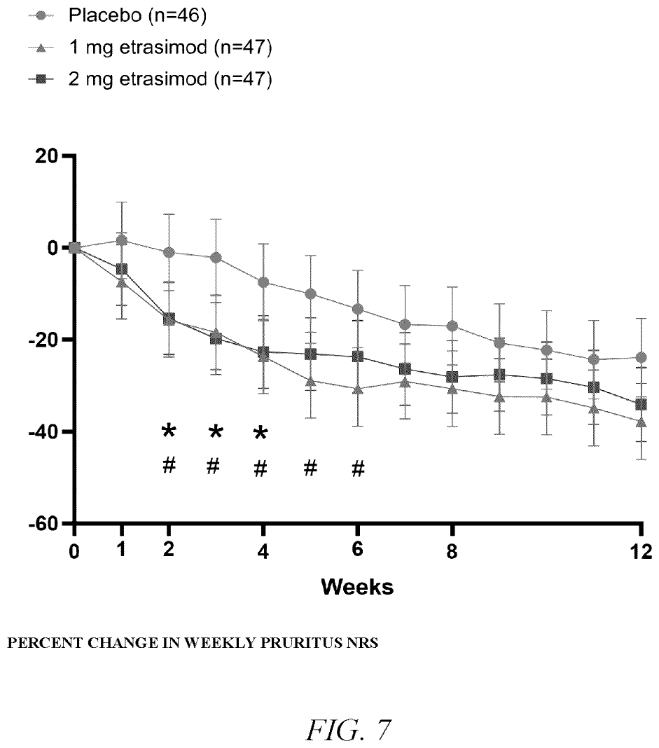

Regarding the pruritis endpoint, the data showed a dose-dependent numerical decrease (improvement) in pruritus greater than or equal to 4 points. The proportion of patients achieving this end point at week 12 were 27.0%, 32.5 (p=0.61) and 42.1 (p=0.15) for placebo, 1 mg and 2 mg etrasimod, respectively. As seen in FIG. 7, the pruritis score decreased in patients receiving 1 mg and 2 mg of etrasimod over 12 weeks of treatment. There was an early and significant effect through week 6 with continued improvement through week 12. A statistically significant improvement in pruritis was observed at week 4 in 2 mg etrasimod dose group.

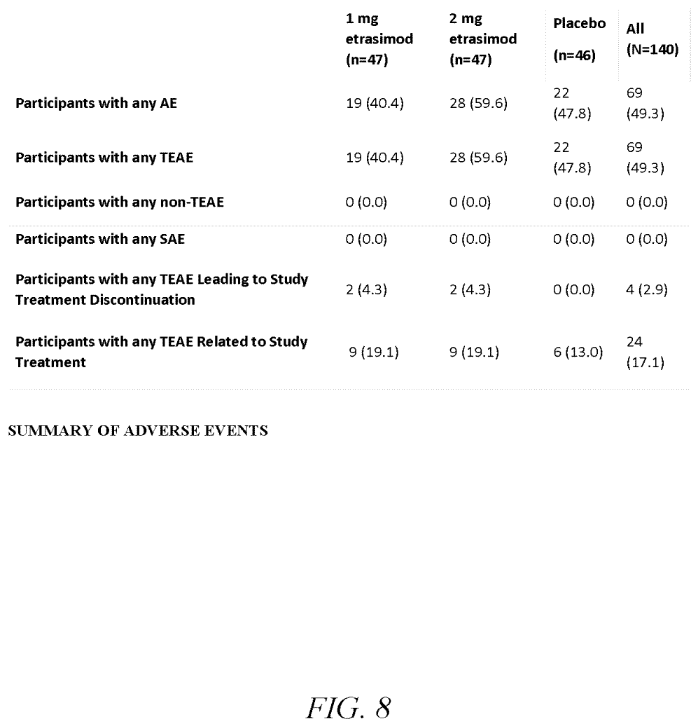

Etrasimod 1 mg and 2 mg was shown to be well tolerated. The adverse event profile is shown in FIG. 8. There were no cases of macular edema, there was one case of dyspnea in the placebo group, and one case in the 2 mg etrasimod treatment group. There were no adverse events related to heart rate. There were no reports of conjunctivitis, acne, venous thromboembolic events, and no opportunistic or serious infections.

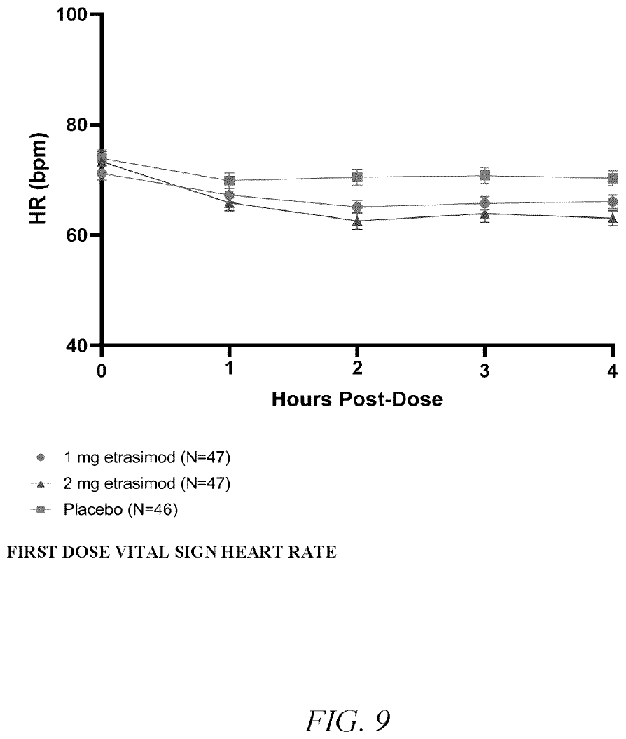

Regarding heart rate effects, as shown in FIG. 9, in evaluating the etrasimod impact on heart rate, a placebo corrected maximal mean heart rate change from baseline was observed at 2 hours. There was a decrease of 1.5 bpm in the 1 mg group and a decrease of 6.7 bpm in the 2 mg group. These heart rate changes were not associated with any blood pressure changes or symptoms. After the first dose, the heart rate differences across the 3 groups came together by week 1 and were similar from weeks 2-12. One participant in the 2 mg etrasimod group had transient 2nd degree AVB Type 1 which was self-limited, and asymptomatic. The participant went on to further dosing without any cardiac changes or symptoms.

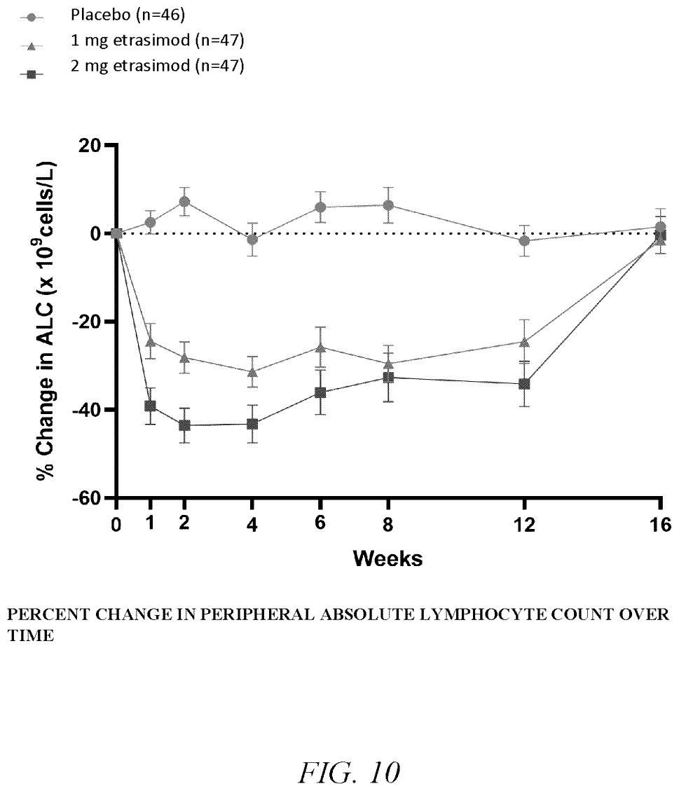

FIG. 10 illustrates the lymphocyte count reductions over the double-blind treatment period. There was a maximal reduction in the peripheral lymphocyte count in the 2 mg etrasimod group of 43.6% at week 2.

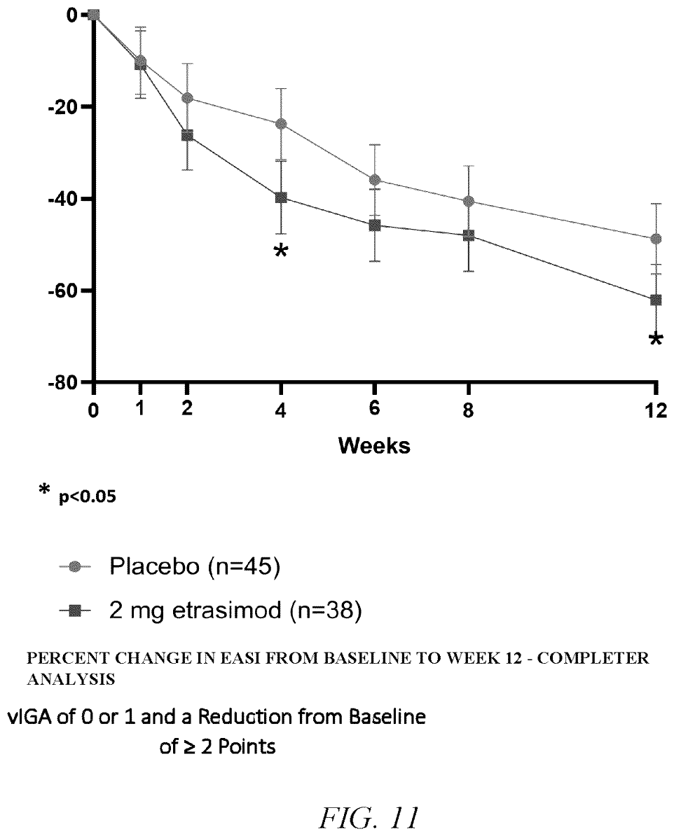

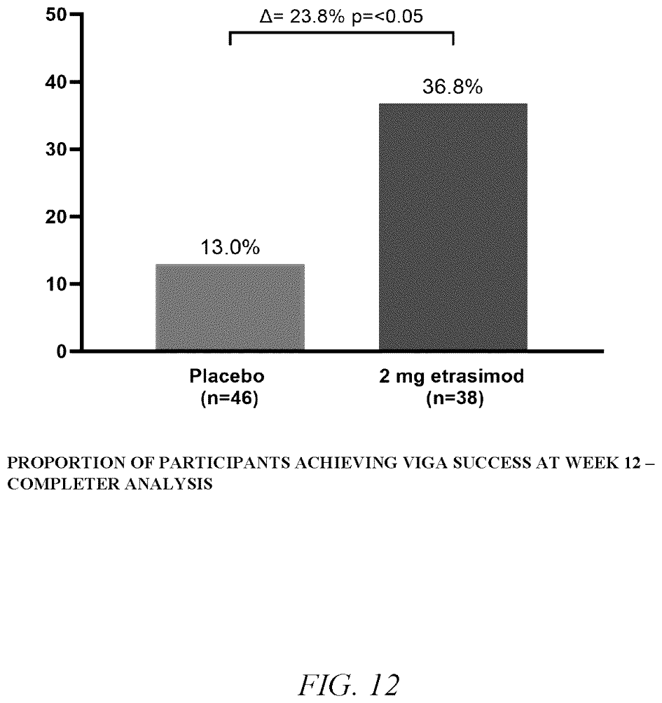

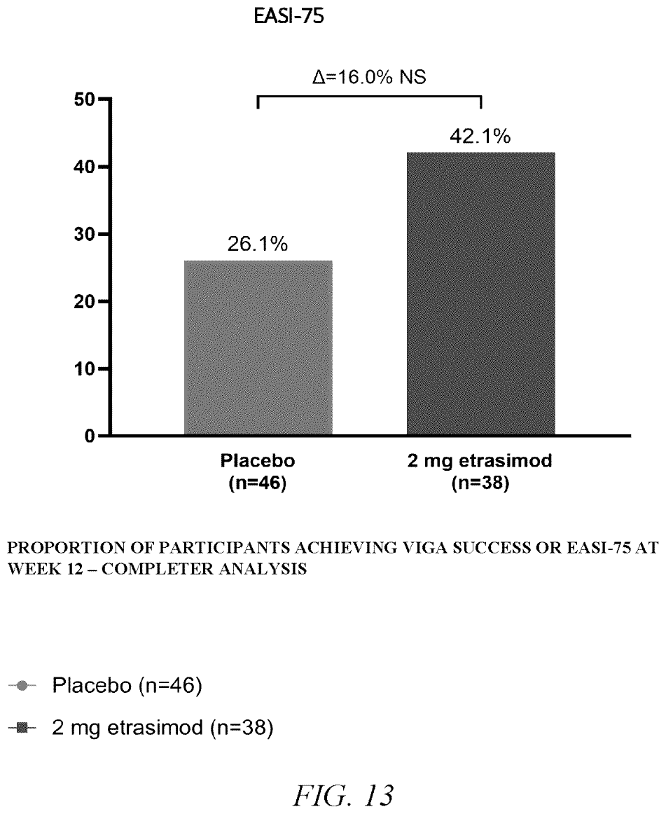

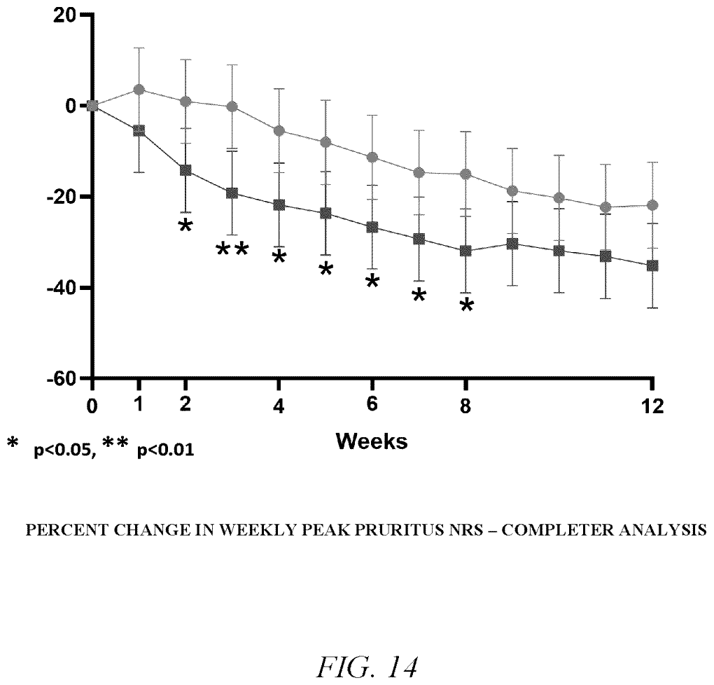

To further understand the impact of the treatment interruptions, the key efficacy measures were also evaluated in the 2 mg etrasimod group that received complete therapeutic exposure (i.e., where the interruptions did not occur), which was 38 participants. As shown in FIG. 11, for the percent change in EASI over time there was clear and early separation in the 2 mg group versus placebo, which was significant at 4 weeks, and continued to show separation out to week 12, which was also statistically significant. The proportion of participants achieving IGA and EASI 75 response at week 12, excluding those participants with dose interruption, were also reanalyzed, and shown at FIGS. 12 and 13. For vIGA, 36.8% of patients in the 2 mg group who did not have study interruptions achieved a vIGA of 0 or 1 and a reduction in vIGA score from baseline of greater than or equal to 2 points, which was statistically significant compared to placebo (delta=23.8%, p<0.05). For EASI 75, 42.1% of the 2 mg etrasimod group who did not have study interruptions achieved EASI-75 at week 12, as compared to 26.1% of patients in the placebo group. As shown in FIG. 14, in reviewing the pruritis NRC changes over time, the etrasimod treatment group when compared to placebo were statistically significant from weeks 2-8.

Other uses of the disclosed methods will become apparent to those in the art based upon, inter alia, a review of this patent document.

1. A method of treating moderate to severe atopic dermatitis comprising:
administering etrasimod, or a pharmaceutically acceptable salt thereof, to a patient in need thereof;
identifying a threshold absolute lymphocyte count (ALC) in the patient;
ceasing administration of the etrasimod, or a pharmaceutically acceptable salt thereof, for an interruption period of time; and
after ceasing administration for the interruption period of time, continuing to administer the etrasimod, or a pharmaceutically acceptable salt thereof, for a continuation period of time;
wherein the patient in need thereof experiences an improvement in moderate to severe atopic dermatitis.

2. The method of claim 1, wherein the improvement in moderate to severe atopic dermatitis comprises an improvement in validated Investigator Global Assessment (vIGA) score, Eczema Area and Severity Index (EASI) score, or body surface area (BSA) score or in an improvement in a patient-reported outcome assessed using the Dermatology Life Quality Index (DLQI), Patient Oriented Eczema Measure (POEM), or Patient Global Assessment (PGA).

3. (canceled)

4. The method of claim 1, wherein the etrasimod, or pharmaceutically acceptable salt thereof, is administered at a frequency of once a day during the continuation period of time.

5. The method of claim 1, wherein the etrasimod, or pharmaceutically acceptable salt thereof, is administered in an amount equivalent to 2 mg or in an amount equivalent to 3 mg of etrasimod.

6. (canceled)

7. The method of claim 1, wherein the threshold ALC is less than 500/mm³ or between 0.2×10e9 cells/L and 0.5×10e9 cells/L.

8. (canceled)

9. The method of claim 1, wherein at least one of the interruption period of time is at least one week or the continuation period of time is at least one month.

10-13. (canceled)

14. The method of claim 1, wherein the patient is diagnosed with moderate atopic dermatitis and has a vIGA score of 3 or the patient is diagnosed with severe atopic dermatitis and has a vIGA score of 4.

15. (canceled)

16. (canceled)

17. The method of claim 1, further comprising the step of testing a circulating blood level of lymphocytes in the patient.

18. (canceled)

19. A method of treating moderate to severe atopic dermatitis comprising:
administering etrasimod, or a pharmaceutically acceptable salt thereof, to a patient in need thereof;
monitoring the patient for one or more infections after administration of the etrasimod or a pharmaceutically acceptable salt thereof;
ceasing administration of the etrasimod, or a pharmaceutically acceptable salt thereof, for an interruption period of time;
after ceasing administration for the interruption period of time, continuing to administer the etrasimod, or a pharmaceutically acceptable salt thereof, for a continuation period of time;
wherein the method is therapeutically effective to treat moderate to severe atopic dermatitis.

20. The method of claim 19, wherein the etrasimod, or pharmaceutically acceptable salt thereof, is administered at a frequency of once a day.

21. (canceled)

22. The method of claim 19, wherein the etrasimod, or pharmaceutically acceptable salt thereof, is administered in an amount equivalent to 2 mg or 3 mg of etrasimod.

23. (canceled)

24. The method of claim 19, wherein at least one of the interruption period of time is at least one week or the continuation period of time is at least one month.

25-28. (canceled)

29. The method of claim 19, where in the patient is diagnosed with moderate atopic dermatitis and a vIGA score of 3 or is diagnosed with severe atopic dermatitis and has a vIGA score of 4.

30. (canceled)

31. The method of claim 19, wherein the patient in need thereof comprises a body surface area (BSA) of atopic dermatitis greater than or equal to 10%.

32. The method of claim 19, further comprising monitoring the patient for infection a subsequent time after the interruption period, and, if an infection is identified, continuing to cease treatment.

33. The method of claim 19, wherein the one or more infections is selected from urinary tract infection, ear infection, common cold, diptheria, E. coli, giardiasis, HIV/AIDS, mononucleosis, influenza, lyme disease, measles, meningitis, mumps, pneumonia, salmonella infections, respiratory infections, shingles, herpes, tuberculosis, viral hepatitis, or COVID-19.

34. The method of claim 1, further comprising:
testing a circulating blood level of lymphocytes in the patient; and
wherein the interruption period of time is one day to four weeks;

35-38. (canceled)

39. A method of treating or ameliorating at least one symptom or indication of atopic dermatitis in an individual in need thereof, comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof, wherein the Compound 1 or pharmaceutically acceptable salt thereof is administered an amount equivalent to more than 2 mg.

40-42. (canceled)

43. The method of claim 39, wherein the improvement to moderate to severe atopic dermatitis comprises an improvement in pruritus.

44-47. (canceled)

48. The method of claim 45, wherein the patient is diagnosed with moderate atopic dermatitis and has a vIGA score of 3 or the patient is diagnosed with severe atopic dermatitis and has a vIGA score of 4.

49. (canceled)

50. (canceled)
